(19) United States
(12) Patent Application Publication
(10) Pub. No.: US 2017/0016889 A1
(43) Pub. Date: Jan. 19, 2017

(54) DEVICE FOR DETECTING AMPLIFIED PRODUCTS OF NUCLEIC ACID

(71) Applicant: GENPRONEX BIOMEDICAL INC., Taipei City (TW)

(72) Inventor: ERR-CHENG CHAN, TAIPEI CITY (TW)

(21) Appl. No.: 14/972,741

(22) Filed: Dec. 17, 2015

(30) Foreign Application Priority Data
Jul. 17, 2015 (TW) 104211552

(51) Int. Cl. G01N 33/543; G01N 33/53

(52) U.S. Cl. CPC G01N 33/54306; G01N 33/5308

(57) ABSTRACT

A device is provided for detecting amplified products of nucleic acid, the device can detect at least one analyte molecule comprising a first marker and a second marker. This device sequentially comprises the following sections along an axial direction: a sample contact section where the analyte molecule is absorbed, a combining section where the analyte molecule is received comprises a reporting carrier specifically bound with the first marker, and a detecting section comprises at least one color reaction section comprising a control unit point having a first combining molecule for specifically binding with the reporting carrier and presenting color, and at least one testing unit point having a second combining molecule for specifically binding with the second marker and presenting color. The control unit point and the testing unit point are separated from each other, and a line connecting them is not parallel to the axial direction.

This application claims priority for Taiwan patent application no. 104211552 filed on Jul. 17, 2015, the content of which is incorporated by reference in its entirely.

### BACKGROUND OF THE INVENTION

Field of the Invention

The present invention relates to a device for detecting amplified products of nucleic acid. More specifically, the present invention discloses the device utilizing molecular immune testing method for simultaneously detecting a plurality of amplified products of nucleic acid.

Description of the Prior Art

To confirm whether the target nucleic acid exists in analyte molecules or not, conventional clinical test method often utilizes different technologies, for instance, polymerase chain reaction (PCR), to amplify the target nucleic acid, and then a technician uses gel electrophoresis or label fluorescence technique to read the test result. Though the technician can macrographically interpret if the target nucleic acid exists in the analyte molecules via the conventional clinical test method, it takes lots of time to experiment, and requires expensive and sophisticated instruments (e.g. fluorophotometer for label fluorescence) as well as professional technicians. Hence, the test result cannot be promptly provided to the technician to attain the goal of point-of-care (POC) testing.

In order to readily obtain the test result, a lateral flow test strip system is invented. Taiwan Patent No. M432833 discloses a rapid testing kit. The testing kit disclosed in this reference comprises a sample section, a combining section, and a display section. The sample section is used for allowing a sample to enter the testing kit and comprises an absorbent substance and a solution absorbed into the absorbent substance. One end of the combining section is connected with one end of the sample section and includes a bonding material. The combining section is used for allowing the sample to selectively combine the bonding material. The display section sequentially comprises a testing zone and a comparing zone. One end of the testing zone is connected to the other end of the combining section and includes a testing display unit coated with a testing substance. One end of the comparing zone is connected to the other end of the testing zone and includes a comparing display unit coated with a comparing substance. In addition, Taiwan patent Patent No. M395826 discloses a Dengue fever test strip. The Dengue fever test strip comprises a support member, and a molecular layer, a reactant releasing layer, a result testing layer, and an absorbing layer, all of which are disposed upon the supporting member, wherein the reactant releasing layer is coated with reactants that can specifically react with analyte molecules. When the analyte molecules are carried to the reactant releasing layer by capillary action of the absorbing layer, the analyte molecules react with the reactants of the reactant releasing layer to form a complex. When the complex passes through a test line and a control line of the result testing layer, the complex reacts with the reactive reagents on the result testing layer, thereby resulting in a color reaction. According to the color reaction, the technician can interpret if the target in the analyte molecules exists. Compared with the conventional clinical test method for reading test result, this lateral flow test strip system has advantages of low cost, easy to operate, fast, and easy to interpret. However, the device applying the lateral flow test strip system usually presents the test result in a striation color block manner, so that it can only be used to detect a single target in the analyte molecules. If it is desired to test different targets in the analyte molecules, different test devices must be employed. If it is desired to test multiple targets with a device applying the aforementioned lateral flow test strip system, several reaction blocks containing the test line and the control line must be set within a limited space in the device. If several color developments happen, the technician may misjudge the test result or had difficulty in interpreting the test result due to the highly concentrated test strips. Moreover, these conventional devices usually employ lots of specific antibodies to detect the test targets. In order to present the test result in striations, a great quantity of expensive specific antibodies must be coated within the test zone, with the test result of a single target being obtained. Obviously, the conventional lateral flow test strip system is not cost-effective, which in turn would result in the waste of precious antibodies.

### SUMMARY OF THE INVENTION

In order to overcome the disadvantages of the conventional lateral flow test strip system that the lateral flow test strip system can detect a single target only or cause confusion when the lateral flow test strip system is used to detect multiple targets simultaneously, with a great quantity of antibodies being wasted.

To this end, the present invention provides a device for detecting amplified products of nucleic acid. The device of the present invention is configured to detect at least one analyte molecule. The analyte molecule comprises a first marker and a second marker being different from the first marker. The device for detecting amplified products of nucleic acid sequentially comprises the following sections along an axial direction: a sample contact section for absorbing the analyte molecule, a combining section connected with the sample contact section and receiving the analyte molecule, and a detecting section. The combining section comprises a reporting carrier which specifically binds with the first marker. The detecting section is connected with the combining section and receives a complex consisting of the analyte molecule and the reporting carrier bound with the first marker. The detecting section comprises at least one color reaction section which comprises a control unit point having a first combining molecule for specifically binding with the reporting carrier for presenting color, and at least one testing unit point having a second combining molecule for specifically binding with the second marker for presenting color, wherein the control unit point and the testing unit point are separated from each other. A connecting line connecting the control unit point and the testing unit point is not parallel to the axial direction.

In one embodiment, the analyte molecule comprises target nucleic acid amplified by nucleic acid amplification technologies. The nucleic acid amplification technologies include polymerase chain reaction (PCR), nucleic acid sequence-based amplification (NASBA), self-sustained sequence replication (3SR), strand displacement amplification (SDA), loop-mediated isothermal amplification (LAMP) or recombinase polymerase amplification (RPA).

In one embodiment, the first marker is biotin.

In one embodiment, the second marker comprises fluorescein isothiocyanate (FITC), digoxigenin (Dig), 5-carboxytetramethylrhodamine (TAMRA), cyanine dyes 3 or cyanine dyes 5.

In one embodiment, the reporting carrier comprises a colloidal gold particle and streptavidin molecules adhered to the surface of the colloidal gold particle.

In one embodiment, the first combining molecule comprises anti-streptavidin antibody or anti-biotin antibody.

In one embodiment, the second combining molecule comprises anti-FITC antibody, anti-Dig antibody, anti-TAMRA antibody, anti-cyanine dyes 3 antibody or anti-cyanine dyes 5 antibody.

In one embodiment, the color reaction section comprises one control unit point and two the testing unit points respectively set at both sides of the control unit point. A connecting line connecting the control unit point and the two testing unit points is not parallel to the axial direction.

In one embodiment, the connecting line connecting the control unit point and the testing unit point is perpendicular to the axial direction.

In one embodiment, the device detects a plurality of analyte molecules, and the second marker on each of the analyte molecule is different from one another. The detecting section comprises a plurality of color reaction sections, and each of the color reaction sections comprises a control unit point having the first combining molecule, and at least one testing unit point having the second combining molecule corresponding to different second marker, wherein the control unit point and the testing unit point are separated from each other.

In one embodiment, the color presented after the reporting carrier and the first combining molecule are combined, and the color presented after the second marker and the second combing molecule are combined, fall into the visible light spectrum.

The number of the color reaction sections in the detecting section of the device for detecting amplified products of nucleic acid of the present invention can be varied depending on the number of analyte molecules to be detected. In every color reaction section, the line connecting the control unit point and the testing unit point is not parallel to the axial direction. Therefore, the internal space of the detecting section can be utilized efficiently so that the goal of detecting a plurality of analyte molecules can be achieved, the testing result of multiple analyte molecules can be interpreted easily, and the amount of antibody used can be reduced. The device for detecting amplified products of nucleic acid according to the present invention employs a particular design by using the control unit point and the testing unit point to present functions of positioning and dual verification (double detection). In addition to the advantage of convenience offered by the conventional lateral flow test strip system, the device of the present invention can detect multiple analyte molecules in a single testing, thereby promoting testing efficiency and interpreting accuracy for providing better disease diagnosis.

### BRIEF DESCRIPTION OF THE DRAWINGS

FIG. 1 is a drawing illustrating the structure of the device for detecting amplified products of nucleic acid according to an embodiment of the present invention;

FIG. 2 is a drawing illustrating the structure of the device for detecting amplified products of nucleic acid according to another embodiment of the present invention; and

FIG. 3-1, FIG. 3-2 and FIG. 3-3 are drawings illustrating the testing flow of the device for detecting amplified products of nucleic acid according to an embodiment of the present invention.

### DESCRIPTION OF THE PREFERRED EMBODIMENTS

The number and shape of molecule in the drawings are only made for illustrating embodiments of the invention. The description serves to explain the principles of the invention rather than limit the scope of the invention and its equivalent.

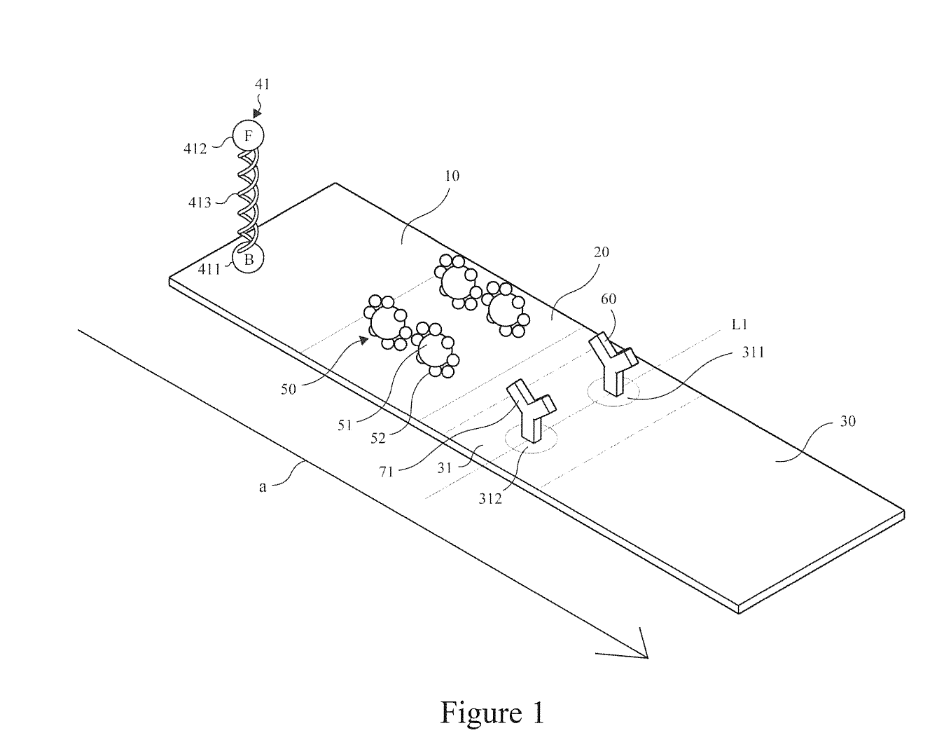

Refer to FIG. 1. The present invention discloses a device for detecting amplified products of nucleic acid. The device is used for detecting at least one analyte molecule 41 in a sample. The analyte molecule 41 comprises a first marker 411 and a second marker 412 being different from the first marker 411. The device sequentially comprises the following sections along an axial direction a: a sample contact section 10 where the analyte molecule 41 is absorbed, a combining section 20 connected with the sample contact section 10 and receiving the analyte molecule 41, and a detecting section 30 connected with the combining section 20. In the present invention, in order to allow the analyte molecule 41 to sequentially pass from the sample contact section 10 through the combining section 20 to the detecting section 30, each section is made of water-absorbing material which can be cotton paper or glass fiber. The water-absorbing material utilizes the capillary action to pass the analyte molecule 41 through the sample contact section 10, the combining section 20 and the detecting section 30. The combining section 20 comprises a reporting carrier 50 which specifically binds with the first marker 411. The detecting section 30 receives a complex of the analyte molecule 41 and the reporting carrier 50 bound with the first marker 411. The detecting section 30 is made of protein-absorbing material, such as nitrocellulose membrane. The detecting section 30 comprises at least one color reaction section 31, which further comprises a control unit point 311 having a first combining molecule 60 for specifically binding with the reporting carrier 50 for presenting color, and at least one testing unit point 312 having a second combining molecule 71 for specifically binding with the second marker 412 for presenting color. The control unit point 311 and the testing unit point 312 are separated from each other, and a connecting line L1 connecting the control unit point 311 and the testing unit point 312 is not parallel to the axial direction a. In the preferred embodiment, the connecting line L1 connecting the control unit point 311 and the testing unit point 312 is perpendicular to the axial direction a.

In the preferred embodiment, the first marker 411 is biotin, and the second marker 412 can be fluorescein isothiocyanate (FITC), digoxigenin (Dig), 5-carboxytetramethylrhodamine (TAMRA), cyanine dyes 3, or cyanine dyes 5. The first marker 411 and the second marker 412 can be other protein or nucleic acid depending on different needs.

In the preferred embodiment, the analyte molecule 41 comprises target nucleic acid 413 amplified by nucleic acid amplification technologies. These nucleic acid amplification technologies comprise polymerase chain reaction (PCR), nucleic acid sequence-based amplification (NASBA), self-sustained sequence replication (3SR), strand displacement amplification (SDA), loop-mediated isothermal amplification (LAMP) or recombinase polymerase amplification (RPA).

In the preferred embodiment, the reporting carrier 50 comprises a colloidal gold particle 51 and streptavidin molecules 52 adhered to surface of the colloidal gold particle 51. The reporting carrier 50 may be other proteins or nucleic acids to bind with the first marker 411 depending on different needs.

In the preferred embodiment, the first combining molecule 60 is anti-streptavidin antibody or anti-biotin antibody. The first combining molecule 60 may be other antibodies, proteins or nucleic acids to specifically bind with the reporting carrier 50 depending on different needs.

In the preferred embodiment, the second combining molecule 71 can be anti-FITC antibody, anti-Dig antibody, anti-TAMRA antibody, anti-cyanine dyes 3 antibody or anti-cyanine dyes 5 antibody. The second combining molecule 71 may be other antibodies, proteins or nucleic acids to specifically bind with the second marker 412 depending on different needs.

For the convenience of interpreting a test result with naked eyes, the color presented after the reporting carrier 50 and the first combining molecule 60 combine with each other and the color presented after the second marker 412 and the second combining molecule 71 combine are within the visible light spectrum.

Referring to FIG. 2 and FIG. 3-1, which illustrate another embodiment of the present invention that the device for detecting amplified products of nucleic acid can detect a plurality of analyte molecules 41, 42, 43. Each of the analyte molecules 41, 42, 43 includes the same first marker 411, 421, 431 but different second marker 412, 422, 432. In the preferred embodiments, the first marker 411, 421, 431 is biotin; the second marker 412, 422, 432 can be one of fluorescein isothiocyanate (FITC), digoxigenin (Dig), 5-carboxytetramethylrhodamine (TAMRA), cyanine dyes 3 or cyanine dyes 5. Based on different testing purposes, the detecting section 30 in the preferred embodiments comprises several color reaction sections 31, 32, 33, and each of the color reaction sections 31, 32, 33 comprises a control unit point 311, 321, 331 having the first combining molecule 60, and at least one testing unit point 312, 322, 332 having the second combining molecule 71, 72, 73 being corresponding to the second marker 412, 422, 432, wherein the control unit point 311, 321, 331 and the testing unit point 312, 322, 332 are separated from each other. In order to improve the credibility of the test result, in the preferred embodiment, the device for detecting amplified products of nucleic acid possesses the function of dual verification (double detection). Each of the color reaction sections 31, 32, 33 preferably comprises one control unit point 311, 321, 331 and two testing unit point 312, 322, 332 respectively set at both sides of the control unit point 311, 321, 331. Connecting lines L1, L2, L3 connecting the control unit point 311, 321, 331 and the testing unit point 312, 322, 332 are not parallel to the axial direction a. In the preferred embodiment, the connecting lines L1, L2, L3 connecting the control unit points 311, 321, 331 and the testing unit points 312, 322, 332 are perpendicular to the axial direction a.

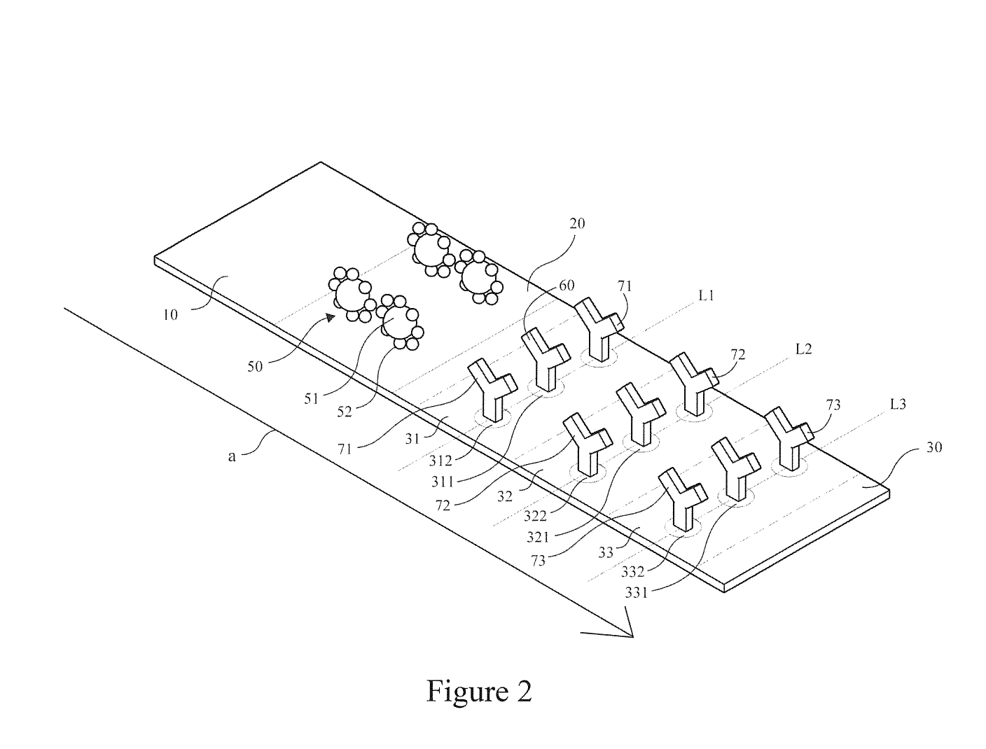

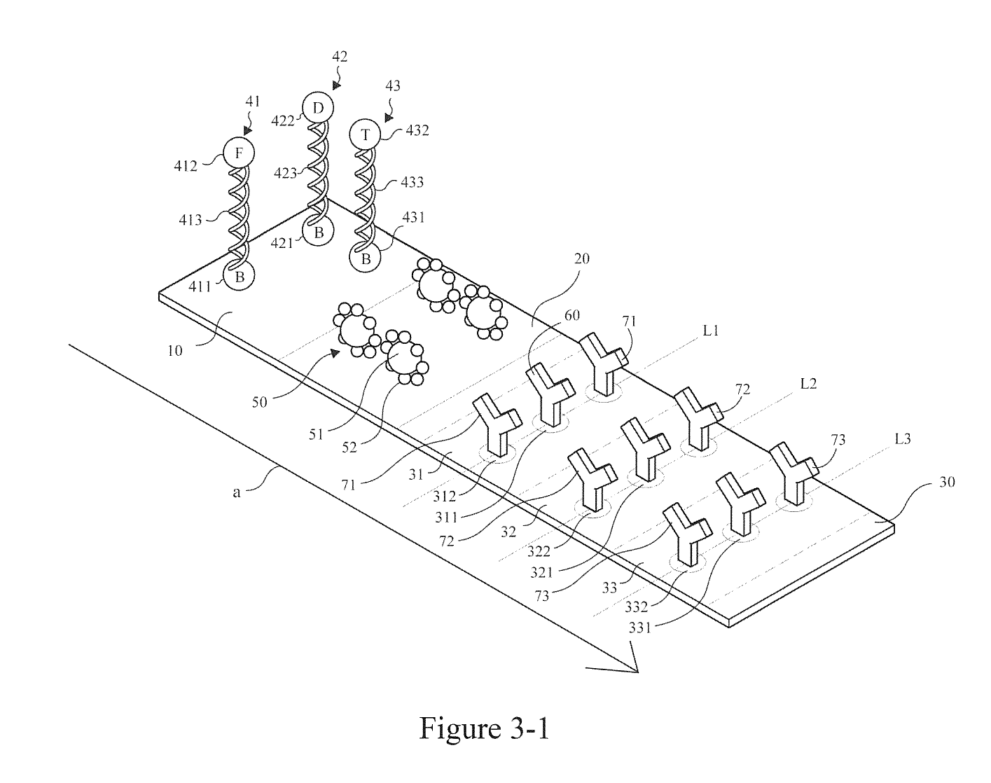

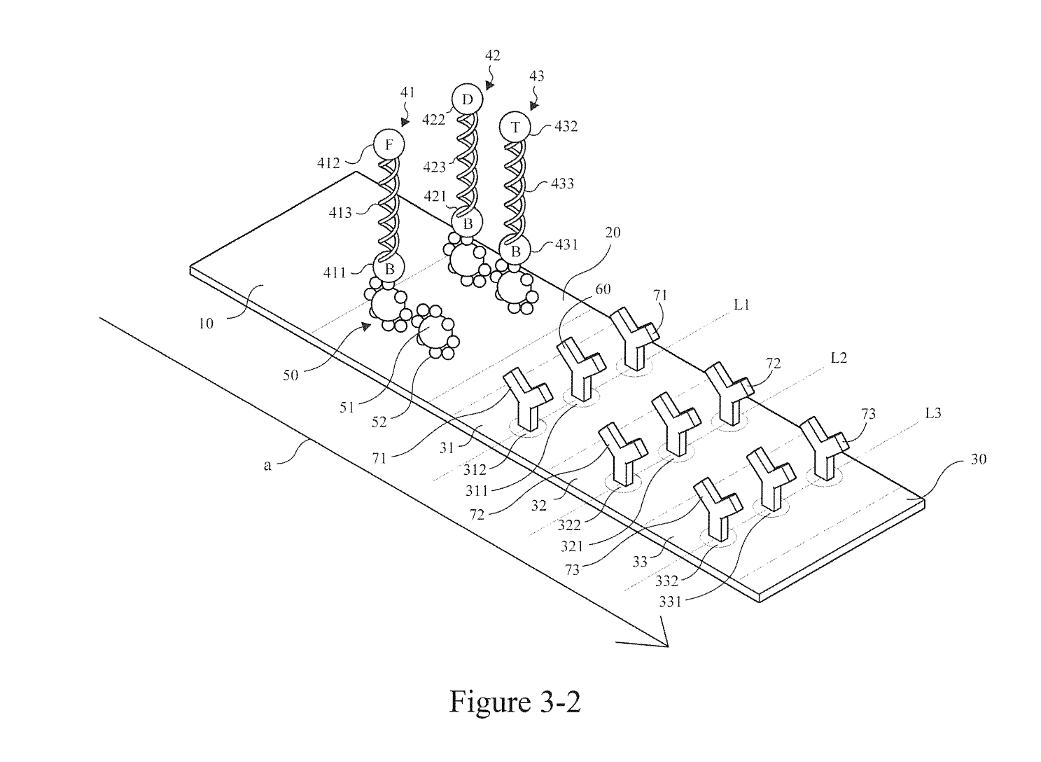

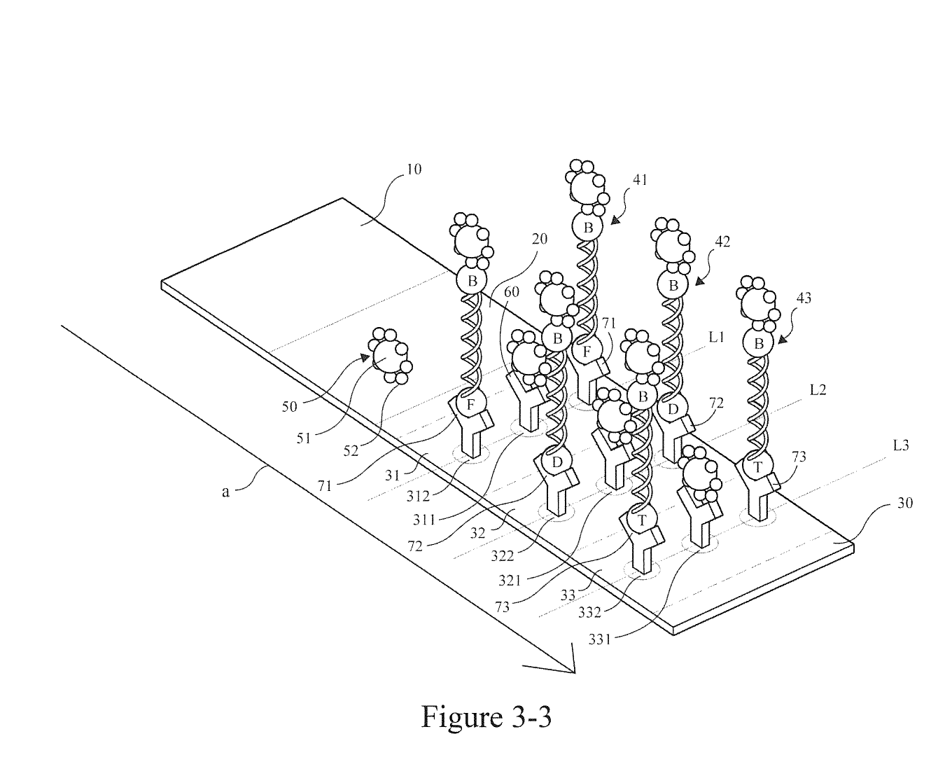

Referring to FIG. 3-1, FIG. 3-2 and FIG. 3-3, which illustrate the steps of detecting a plurality of the analyte molecules 41, 42, 43 by the device for detecting amplified products of nucleic acid of the present invention. In the preferred embodiments, a sample is to be detected to see if specific analyte molecules 41, 42, 43, such as nucleic acids, exist therein. In order to increase detection sensitivity and coat a first marker 411, 421, 431 and a second marker 412, 422, 432 on the analyte molecules 41, 42, 43, the sample has to undergo the DNA purification process, and then the nucleic acid amplification technology, such as PCR or RPA, is used to amplify the analyte molecules 41, 42, 43. That is, the first marker 411, 421, 431 and the second marker 412, 422, 432 are labeled at the 5′ end and 3′ end of the target nucleic acid 413, 423, 433 in the analyte molecules 41, 42, 43. The first marker 411, 421, 431 is biotin, and the second marker 412, 422, 432 can be fluorescein isothiocyanate (FITC), digoxigenin (Dig) and 5-carboxytetramethylrhodamine (TAMRA). After the nucleic acid amplification process is completed, the analyte molecules 41, 42, 43 are dripped into the sample contact section 10, as shown in FIG. 3-1. The analyte molecules 41, 42, 43 move to the combining section 20 via capillary action, and then form several complexes by binding the first marker 411, 421, 431 with the reporting carriers 50, as shown in FIG. 3-2. In the preferred embodiment, the reporting carrier 50 comprises a colloidal gold particle 51 and streptavidin molecules 52 adhered to the surface of the colloidal gold particle 51. The first marker 411, 421, 431 on the analyte molecules 41, 42, 43, i.e. the biotin, would bind with the streptavidin molecules 52 on the reporting carrier 50.

The complexes of the analyte molecules 41, 42, 43 and the reporting carriers 50 bound with the first marker 411, 421, 431, and the reporting carriers 50 that are not bound with the analyte molecules 41, 42, 43, continue moving towards the detecting section 30, as shown in FIG. 3-3. The detecting section 30 comprises three color reaction sections 31, 32, 33, each of which comprises a control unit point 311, 321, 331 and two testing unit points 312, 322, 332 respectively set at both sides of the control unit point 311, 321, 331. The control unit point 311, 321, 331 comprises the first combining molecule 60, and the testing unit points 312, 322, 332 respectively comprise the second combining molecule 71, 72, 73, which corresponds to the different kinds of the second marker 412, 422, 432. The first combining molecule 60 on the control unit point 311, 321, 331 is anti-streptavidin antibody. The second combining molecules 71, 72, 73 on the testing unit points 312, 322, 332 are anti-FITC antibody, anti-Dig antibody, and anti-TAMRA antibody. After the complexes of the analyte molecules 41, 42, 43 and the reporting carriers 50 bound with the first marker 411, 421, 431, and the reporting carriers 50 that are not bound with the analyte molecules 41, 42, 43 enter the detecting section 30, the first combining molecule 60 on the control unit point 311, 321, 331 binds with the streptavidin molecules 52 on the reporting carrier 50, and then precipitate. The colloidal gold particle 51 in the reporting carrier 50 is red. Therefore, the control unit point 311, 321, 331 is manifested with a red dot. The second combining molecules 71, 72, 73 on the testing unit points 312, 322, 332 respectively bind with the second markers 412, 422, 432 on the analyte molecules 41, 42, 43, and then precipitate. A technician may interpret the result according to the color presented after the second markers 412, 422, 432 and the colloidal gold particles 51 combine with each other. The red dot presented at the control unit point 311, 321, 331 may serve as the control group to determine whether the reporting carrier 50 has reached the color reaction sections 31, 32, 33, and may function as a positioning mark for the analyte molecules 41, 42, 43 in the same color reaction sections 31, 32, 33, so that the technician can easily observe and distinguish the test results of different analyte molecules 41, 42, 43. If the target analyte molecules 41, 42, 43 exist in the sample, the testing unit points 312, 322, 332 in the color reaction sections 31, 32, 33 present colored dots. If target analyte molecules 41, 42, 43 do not exist in the sample, the testing unit points 312, 322, 332 in the color reaction sections 31, 32, 33 do not present any color.

The detecting section of the device for detecting amplified products of nucleic acid of the present invention can set up a number of color reaction sections in compliance with the number of target analyte molecules. In every color reaction section, a line connecting the control unit point and the testing unit point is not parallel to the axial direction. Consequently, the internal space of the detecting section can be utilized efficiently to fulfill the purposes of detecting a plurality of analyte molecules, easily interpreting the test result of multiple analyte molecules, and reducing the antibody usage. The device for detecting amplified products of nucleic acid of the present invention employs a particular design of the control unit point and the testing unit point to fulfill functions of positioning and dual verification (double detection). In addition to the advantage of convenience offered by the conventional lateral flow test strip system, the device of the present invention can detect multiple analyte molecules in single testing. This feature can significantly promote testing efficiency and interpreting accuracy.

What is claimed is:

1. A device for detecting amplified products of nucleic acid in at least one analyte molecule, the analyte molecule comprises a first marker and a second marker being different from the first marker, sequentially along an axial direction of the device, the device comprising:
a sample contact section absorbing the analyte molecule;
a combining section connected with the sample contact section and receiving the analyte molecule, the combining section comprising a reporting carrier specifically bound with the first marker; and
a detecting section connected with the combining section and receiving a complex of the analyte molecule and the reporting carrier bound with the first marker, the detecting section comprising at least one color reaction section comprising a control unit point having a first combining molecule for specifically binding with the reporting carrier and presenting color and comprising at least one testing unit point having a second combining molecule for specifically binding with the second marker and presenting color, wherein the control unit point and the testing unit point are separated from each other, and a line connecting the control unit point and the testing unit point is not parallel to the axial direction.

2. The device of claim 1, wherein the analyte molecule comprises a target nucleic acid amplified by a nucleic acid amplification technology.

3. The device of claim 1, wherein the nucleic acid amplification technology comprises polymerase chain reaction (PCR), nucleic acid sequence-based amplification (NASBA), self-sustained sequence replication (3SR), strand displacement amplification (SDA), loop-mediated isothermal amplification (LAMP) or recombinase polymerase amplification (RPA).

4. The device of claim 1, wherein the first marker is biotin.

5. The device of claim 1, wherein the second marker comprises fluorescein isothiocyanate (FITC), digoxigenin (Dig), 5-carboxytetramethylrhodamine (TAMRA), cyanine dyes 3. or cyanine dyes 5.

6. The device of claim 1, wherein the reporting carrier comprises a colloidal gold particle and streptavidin molecules adhered to a surface of the colloidal gold particle.

7. The device of claim 1, wherein the first combining molecule comprises anti-streptavidin antibody or anti-biotin antibody.

8. The device of claim 1, wherein the second combining molecule comprises anti-fluorescein isothiocyanate (FITC) antibody, anti-digoxigenin (Dig) antibody, anti-5-carboxytetramethylrhodamine (TAMRA) antibody, anti-cyanine dyes 3 antibody, or anti-cyanine dyes 5 antibody.

9. The device of claim 1, wherein the color reaction section comprises one the control unit point and two the testing units point respectively set at both sides of the control unit point, a line connecting the control unit point and two testing unit points is not parallel to the axial direction.

10. The device of claim 1, wherein the line connecting the control unit point and the testing unit point is perpendicular to the axial direction.

11. The device of claim 1, wherein:
when the device detects a plurality of analyte molecules, the second marker on each of the analyte molecule is different from one another; and
wherein the detecting section comprises several color reaction sections, and each of the color reaction sections comprises a control unit point having the first combining molecule and at least one testing unit point having the second combining molecule corresponding to different kinds of the second marker, and the control unit point and the testing unit point are separated from each other.

12. The device of claim 1, wherein the color presented after the reporting carrier and the first combining molecule combine with each other and the color presented after the second marker and the second combing molecule combine with each other are within a visible light spectrum.
